(19) United States
(12) Patent Application Publication
(10) Pub. No.: US 2013/0158392 A1
(43) Pub. Date: Jun. 20, 2013

(54) Reciprocating Drive Optical Scanner for Surgical Endoprobes

(71) Applicants: Michael Papac, North Tustin, CA (US); John Christopher Huculak, Mission Viejo, CA (US); Michael J. Yadlowsky, Sunnyvale, CA (US)

(72) Inventors: Michael Papac, North Tustin, CA (US); John Christopher Huculak, Mission Viejo, CA (US); Michael J. Yadlowsky, Sunnyvale, CA (US)

(21) Appl. No.: 13/692,280

(22) Filed: Dec. 3, 2012

Related U.S. Application Data
(60) Provisional application No. 61/577,371, filed on Dec. 19, 2011.

(51) Int. Cl. A61F 9/008; A61B 5/00

(52) U.S. Cl. CPC A61F 9/008; A61F 9/00821; A61B 5/0066
USPC 600/425; 606/4; 606/13; 606/17

(57) ABSTRACT

A microsurgical endoprobe and method for use are provided. The microsurgical endoprobe may have a cannula assembly with a proximal element and a distal element coupled to a mechanical actuator. The microsurgical endoprobe may be configured to rotate at least one of the proximal element and the distal element in a first direction for an arc period, perform a microsurgical procedure on the tissue, and rotate the at least one of the proximal element and the distal element in a second direction opposite to the first direction for the arc period. The microsurgical endoprobe may include a hand-piece having a motor with a cannula assembly coupled to the hand-piece.

### PRIORITY CLAIM

This application claims the benefit of priority of U.S. Provisional Patent Application Ser. No. 61/577,371 titled “Reciprocating Drive Optical Scanner for Surgical Endoprobes”, filed on Dec. 19, 2011, whose inventors are Michael D. Papac, John C. Huculak, and Michael J. Yadlowsky, which is hereby incorporated by reference in its entirety as though fully and completely set forth herein.

### BACKGROUND

1.- Field of the Invention

Embodiments described herein relate to the field of microsurgical probes. More particularly, embodiments described herein are related to the field of endoscopic Optical Coherence Tomography (OCT) and to the field of ophthalmic microsurgical techniques.

2.- Description of Related Art

The field of microsurgical procedures is evolving rapidly. Typically, these procedures involve the use of probes that are capable of reaching the tissue that is being treated or diagnosed. Scanning mechanisms that allow time-dependent direction of light for diagnostic or therapeutic purposes have been used in endoscopic surgical instruments. These instruments typically use probes that provide imaging, treatment, or both, over an extended area of tissue limiting motion of the endoscope relative to its surroundings. This is particularly true for forward-directed scanning probes that may require counter rotating shafts with fixed or controlled relative speeds.

Such procedures may make use of a probe including rotating components in direct contact with ocular tissue and vitreous humor. This may create the problem of tear and stress induced in the tissue or vitreous humor through viscous drag of the rotating element in the probe. The damage produced in the eye by such moving components may be severe, including retinal detachment. To avoid this, prior art approaches may make use of a fixed, external tube shrouding the moving elements in the probe. However, this approach may unnecessarily increase the total cross-section of the probe. In ophthalmic surgery, dimensions of one (1) millimeter (mm) or less are preferred, to access areas typically involved without damaging unrelated tissue. Larger probes require larger incisions and thus complicate surgical procedures and after-surgery recovery. Furthermore, given a probe size that is surgically tolerable, it may be desirable to utilize the entire probe cross-section for the collection optics.

### SUMMARY

According to embodiments disclosed herein, a method for using a microsurgical endoprobe having a cannula assembly, includes: inserting the microsurgical endoprobe into a tissue, the microsurgical endoprobe having a cannula assembly with a proximal element and a distal element coupled to a mechanical actuator; and rotating at least one of the proximal element and the distal element in a first direction for an arc period. The method further includes performing a microsurgical procedure on the tissue; and rotating the at least one of the proximal element and the distal element in a second direction opposite to the first direction for the arc period.

According to further embodiments disclosed herein, a microsurgical endoprobe includes a hand-piece having a motor; and a cannula assembly coupled to the hand-piece. The cannula assembly including: a first tube able to rotate about a longitudinal axis; a second tube within the first tube, able to rotate within the first tube; a distal optical element attached to the first tube; and a proximal optical element attached to the second tube; wherein the motor is coupled to provide a reciprocating rotational motion to at least one of the second tube and the first tube for an arc period.

According to further embodiments in the present disclosure an optical cannula assembly includes an outer tube able to rotate about a longitudinal axis; an inner tube within the outer tube, able to rotate within the outer tube; and a distal optical element attached to the outer tube. The cannula assembly further includes a proximal optical element attached to the inner tube; wherein a proximal end of the cannula assembly is configured to engage a mechanical actuator to provide a reciprocating rotational motion to at least one of the outer tube and the inner tube for an arc period.

These and other embodiments of the present invention will be described in further detail below with reference to the following drawings.

### BRIEF DESCRIPTION OF THE DRAWINGS

FIG. 1A illustrates a microsurgical endoprobe according to some embodiments.

FIG. 1B illustrates a partial cross-sectional view of the microsurgical endoprobe of FIG. 1A along a sagittal plane, according to some embodiments.

FIG. 2A illustrates a rotational mechanism in a microsurgical endoprobe, according to some embodiments.

FIG. 2B illustrates a further rotational mechanism in a microsurgical endoprobe, according to some embodiments.

FIG. 2C illustrates a scanning trajectory of an optical beam, according to some embodiments.

FIG. 3A illustrates a scanning trajectory of an optical beam, according to some embodiments.

FIG. 3B illustrates a scanning trajectory of an optical beam, according to some embodiments.

FIG. 3C illustrates a scanning trajectory of an optical beam, according to some embodiments.

FIG. 4 illustrates a scanning trajectory of an optical beam, according to some embodiments.

FIG. 5 illustrates a chart for a rotational mechanism in a microsurgical endoprobe, according to some embodiments.

FIG. 6 illustrates a chart for a rotational mechanism in a microsurgical endoprobe, according to some embodiments.

FIG. 7 illustrates a chart for a rotational mechanism in a microsurgical endoprobe, according to some embodiments.

FIG. 8 illustrates a flow diagram for a method to implement a rotational mechanism for a microsurgical endoprobe, according to some embodiments.

FIG. 9 illustrates an eccentric configuration for a rotational mechanism in a microsurgical endoprobe, according to some embodiments.

FIG. 10 illustrates a scanning trajectory of an optical beam, according to some embodiments.

In the figures, elements having the same reference number have the same or similar functions.

### DETAILED DESCRIPTION

Microsurgical procedures using endoscopic instruments may include a probe having a simple and cost-effective drive coupling mechanism. The probe may be a hand-held probe, for direct manipulation by specialized personnel. In some embodiments, the probe may be designed to be controlled by a robotic arm or a computer-controlled device. Probes have a proximal end close to the operation controller (be it a specialist or a device), and a distal end, close to or in contact with the tissue. Probes according to embodiments disclosed herein may have small dimensions, be easy to manipulate from a proximal end, and minimally invasive to the surrounding tissue. In the distal end, the probe ends with a tip, from where the probe performs certain actions on a target tissue located in the vicinity of the tip. For example, the probe may deliver light from its tip, and receive light reflected or scattered from the tissue, coupled through the tip. The tip of the probe may include movable elements that enable the tip to perform its action.

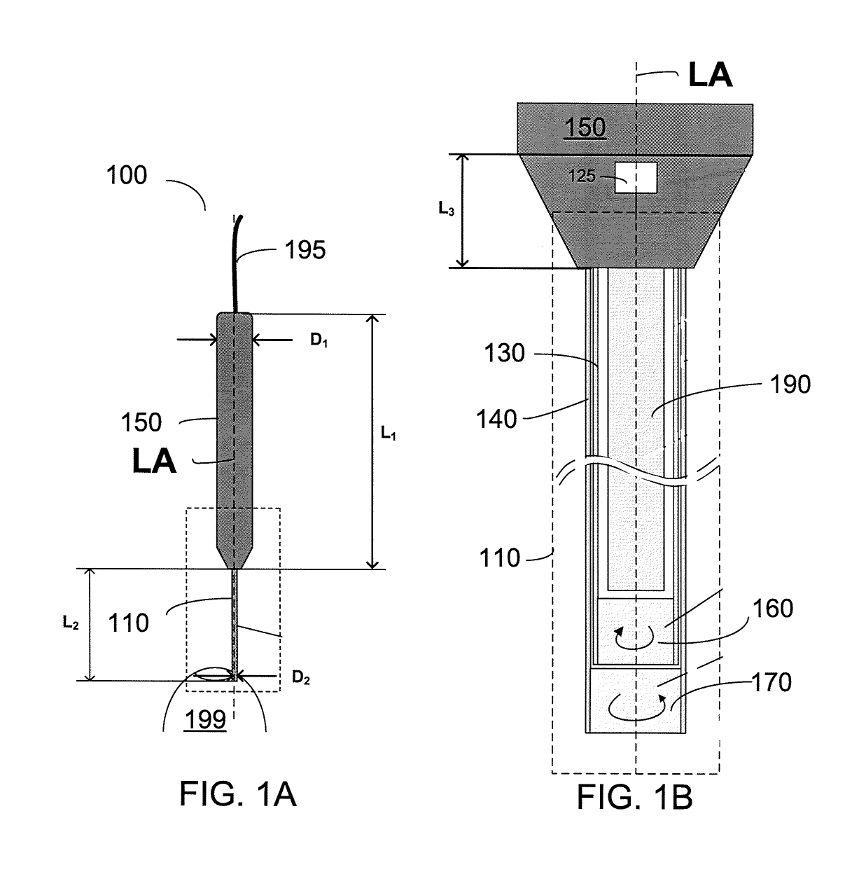

FIG. 1A shows microsurgical endoprobe 100 including optical scanning element 110, hand-piece 150, and coupling cable 195, according to some embodiments. Scanning element 110 may also be referred to as a “cannula assembly” according to some embodiments. Element 110 includes the distal end of endoprobe 100 which may be elongated along the probe longitudinal axis (LA) and have a limited cross-section. For example, in some embodiments cannula assembly 110 may be about 0.5 mm in diameter (D₂) while hand-piece 150 may have a substantially cylindrical shape of several millimeters (mm) in diameter (D₁) such as 12-18 mm.

In some embodiments, assembly 110 may be inserted into an organ, like eye 199. In such configuration, assembly 110 is in contact with tissue, including target tissue for the microsurgical procedure. Thus, assembly 110 may be coated with materials that prevent infection or contamination of the tissue. Furthermore, surgical procedures and protocols may establish hygienic standards for assembly 110. For example, it may be desirable that assembly 110 be disposed of after used once. In some situations, assembly 110 may be disposed of at least every time the procedure is performed on a different patient, or in a different part of the body.

Embodiments of endoprobe 100 and assembly 110 may comply with industry standards such as EN ISO 14971 (2007), “Medical Devices-Application of Risk Management to Medical Devices;” ISO/TS 20993 (2006), “Biological evaluation of medical devices- Guidance on a risk management process;” ISO 14001 (2004), “Environmental management systems—Requirements with guidance for use;” ISO 15752 (2009), “Ophthalmic instruments—endoilluminators—fundamental requirements and test methods for optical radiation safety;” and ISO 15004-2 (2007), “Ophthalmic instruments—fundamental requirements and test methods—Part 2: Light Hazard Protection.”

Hand-piece 150 may be closer to the proximal end of the probe, and may have a larger cross section as compared to element 110. Element 150 may be adapted for manual operation of endoprobe 100, according to some embodiments. Element 150 may be adapted for robotic operation or for holding by an automated device, or a remotely operated device. While assembly 110 may be in contact with living tissue in some applications, element 150 may not be in direct contact with living tissue. Thus, element 150 may comply with hygienic standards somewhat relaxed as compared to those used for assembly 110. For example, element 150 may include parts and components of endoprobe 100 that may be used repeatedly before disposal.

Thus, some embodiments of endoprobe 100 as disclosed herein may include complex components in element 150, and less expensive, replaceable components may be included in assembly 110. Some embodiments may have a removable element 110 which is disposable, while hand-piece 150 may be used more than once. Hand-piece 150 may be sealed hermetically, in order to avoid tissue contamination with particulates or fumes emanating from internal elements in hand-piece 150. In some embodiments, cannula assembly 110 may be fixed to hand-piece 150 by an adhesive bonding. According to other embodiments, assembly 110 may be removable from hand-piece 150 to allow easy replacement of assembly 110 for repeated procedures. Some embodiments consistent with FIG. 1A may have a disposable element 150 and a disposable assembly 110.

Cable 195 may be included in some embodiments to couple endoprobe 100 to a remote console or controller device. Cable 195 may include power transmission elements, to transfer electrical or pneumatic power to a mechanical actuator, or motor inside element 150. Cable 195 may include transmission elements to carry optical information and power, such as a laser beam, from a remote console or controller, to the tissue. An optical transmission element may also carry optical information from the tissue to a remote console or controller, for processing. For example, cable 195 may include at least one or more optical fibers to transmit light to and from the tissue. In some embodiments, one optical fiber may transmit light to the tissue, and another optical fiber may transmit light from the tissue. Further, some embodiments may transmit light to and from the tissue through one optical fiber.

According to some embodiments of endoprobe 100, cable 195 may be absent, and the probe may be wirelessly accessible. In such embodiments, a battery may be included in hand-piece 150 to provide electrical power to a motor and an optical light source. Further, in embodiments where hand-piece 150 is wireless, hand-piece 150 may include a transceiver device to send and receive data and instructions from the probe to a controller, and vice versa. In such embodiments, hand-piece 150 may also include a processor circuit having a memory circuit to process data, control assembly 110, and control the transceiver device.

FIG. 1B shows a partial cross-sectional detail of microsurgical endoprobe 100 in FIG. 1A including motor 125, inner tube 130 and outer tube 140, according to some embodiments. Also shown in FIG. 1B are proximal element 160, coupled to inner tube 130, distal element 170, coupled to outer tube 140, and optical transmission element 190. Transmission element 190 may include an optical fiber, or a plurality of optical fibers. As described above, element 190 may be coupled to cable 195 in the proximal end of assembly 110, and may transmit light into and from the tissue. In some embodiments, the outer tube has a first cross-sectional area and the distal optical element has a second cross-sectional area such that the second cross-sectional area is greater than the first cross-sectional area (e.g., greater than 50% of the first cross-sectional area, greater than 60% of the first cross-sectional area, greater than 70% of the first cross-sectional area, etc.)

In some embodiments, motor 125 may be an electric motor or a linear actuator. Embodiments consistent with the present disclosure may include continuous electric motors and stepper motors. Some embodiments may include motors that use fluid flows to produce motion. For example, a pneumatic actuator may be used as motor 125 in embodiments consistent with FIGS. 1A and 1B. A pneumatic actuator in motor 125 may include a piston mechanism or a fan, according to some embodiments. In some embodiments, motor 125 includes a piezo-electric actuator. A piezoelectric motor used in embodiments consistent with the present disclosure include ratchet-type piezoelectric motors, and continuous-type piezoelectric motors driven by high frequency vibrations. According to embodiments disclosed herein, motor 125 provides a high torque for rotating inner tube 130 and outer tube 140, and reversing the rotation in a reciprocating motion. Motor 125 may include an encoder to provide an indication of the position of a rotating shaft within the motor at every point in time. The encoder may be coupled to the controller in a remote console through cable 195, or wirelessly, according to some embodiments.

Further embodiments of motor 125 may be as disclosed in detail in U.S. Provisional Patent Application No. 61/466,364 entitled “Pneumatically Driven Ophthalmic Scanning Endoprobe” by Michael J. Papac, Michael Yadlowsky, and John Huculak, filed on Mar. 22, 2011, which is incorporated by reference in its entirety as though fully and completely set forth herein. Also, embodiments of counter-rotating mechanisms for cannula assemblies may be as disclosed in detail in U.S. Provisional Patent Application No. 61/434,942 entitled “Counter-rotating Ophthalmic Scanner Drive Mechanism,” by Michael Yadlowsky, Michael J. Papac, and John Huculak, filed on Jan. 21, 2011 which is incorporated by reference in its entirety as though fully and completely set forth herein. Some embodiments consistent with the present disclosure may use a motor 125 according to embodiments described in detail in U.S. Provisional Patent Application No. 61/577,379, filed on Dec. 19, 2011, entitled “Concentric Drive Scanning Probe,” by Michael J. Papac, Michael Yadlowsky, and John C. Huculak, incorporated by reference in its entirety as though fully and completely set forth herein.

According to embodiments consistent with FIG. 1B, inner tube 130 may be aligned with its symmetry axis along the probe longitudinal axis (LA). Inner tube 130 may be a hollow tube of a material that provides rigidity to assembly 110 and support to element 160. Proximal element 160 may be attached to inner tube 130. Element 160 may be an optical element, according to some embodiments configured for microsurgical procedures. For example, in forward-scan OCT techniques, element 160 may include a lens having one of its flat ends cut at a predetermined angle relative to the optical axis of the lens. In some embodiments, the lens may be arranged with its optical axis along the probe LA, with its angled end on the distal side of the lens. In some embodiments the lens in element 160 may be a GRIN (gradient index) lens.

According to embodiments consistent with FIG. 1B, outer tube 140 may include a rotating cannula tube coupled to distal element 170. Tube 140 may be a hollow tube of a material that provides rigidity to assembly 110 and support to element 170, aligned with its symmetry axis along the probe LA. Element 170 may be an optical element, according to some embodiments configured for microsurgical procedures. For example, in forward-scan OCT techniques, element 170 may include a lens having one of its flat ends cut at a predetermined angle relative to the optical axis of the lens. In some embodiments the lens in element 170 may be a GRIN lens. In some embodiments, the GRIN lens may be arranged with its optical axis along the probe LA, having an angled end on the proximal side.

The reference to inner tube 130 as “rotating” and outer tube 140 as “counter-rotating” is arbitrary and establishes the relative motion between tubes 130 and 140 about the probe LA. In some embodiments, while tube 130 rotates ‘clockwise’ with respect to a fixed point in the surrounding tissue, tube 140 may rotate ‘counter-clockwise’ with respect to the fixed point. The opposite configuration may occur, wherein tube 130 rotates ‘counter-clockwise’ and tube 140 rotates ‘clockwise.’

The materials used to form cannula elements 130 and 140 may be any of a variety of biocompatible materials. For example, some embodiments may include elements 130 and 140 made of stainless steel, or plastic materials. Furthermore, some embodiments may have a portion or the entirety of elements 130 and 140 coated with a protective layer. The coating material may be a gold layer, or a biocompatible polymer. In some embodiments the role of the coating layer may be to provide lubrication and friction relief to moving parts in assembly 110. For example, coating materials may reduce friction between the inner face of tube 140 and the outer face of tube 130. In some embodiments the role of the coating layer may be to provide protection to the tissue in direct contact with assembly 110. For example, in some embodiments outer tube 140 may include a tissue engaging surface suitably coated to reduce friction, abrasion, or contamination of tissue. In some embodiments the tissue engaging surface may be coated with gold.

Table I illustrates a range of dimensions of different elements as labeled in FIGS. 1A and 1B according to some embodiments. In Table I, ‘ID’ refers to inner diameter, and ‘OD’ refers to outer diameter. Units in Table I are in microns (1 μm=10⁻⁶ m). The dimensions provided in Table I are nominal and can vary in different embodiments depending on the specific application. For example, some embodiments may vary endoprobe dimensions by about 50% from those in Table I. In some embodiments of assembly 110 configured for ophthalmic microsurgical procedures ‘ODs’ of less than approximately 1 to 1.5 mm may be used.

TABLE I

| Element | OD max | OD min | ID max | ID min |
|---|---|---|---|---|
| 140 | 546.1 | 533.4 | 495.3 | 469.9 |
| 130 | 419.1 | 406.4 | 381 | 355.6 |
| 190 | 342.9 | 330.2 | 152.4 | 139.7 |

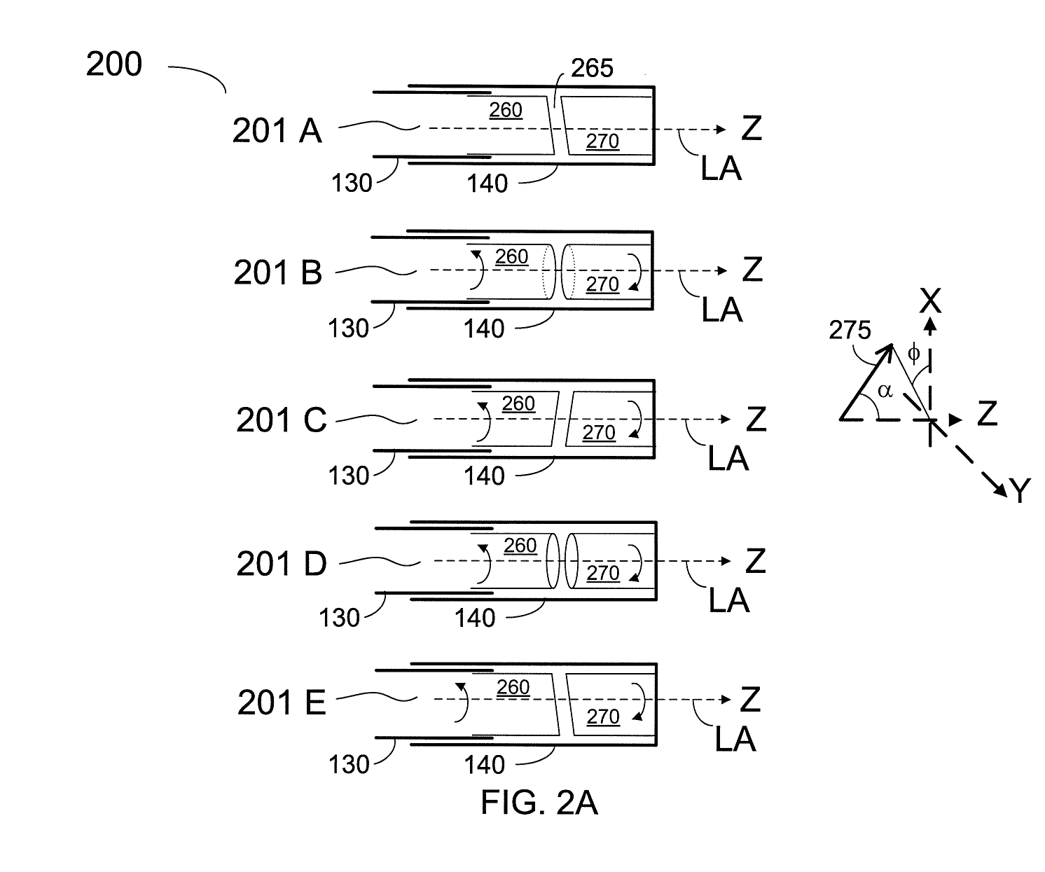

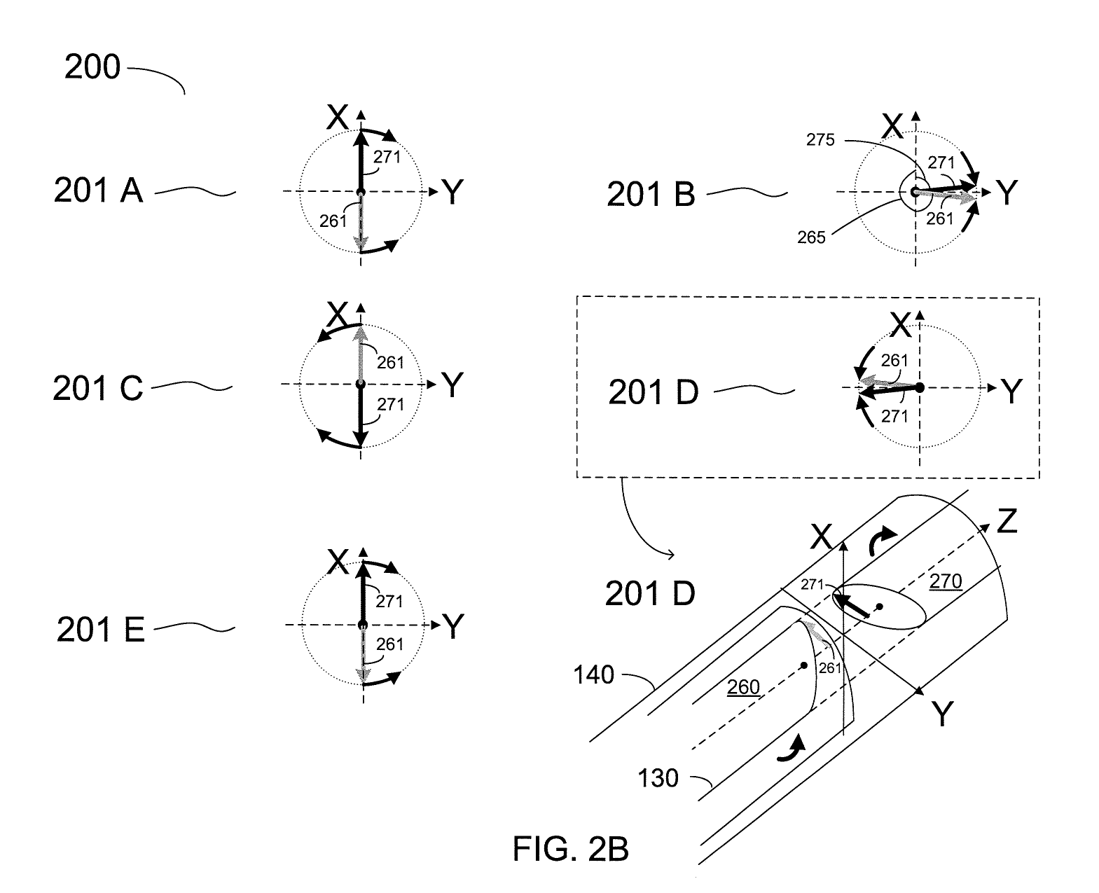

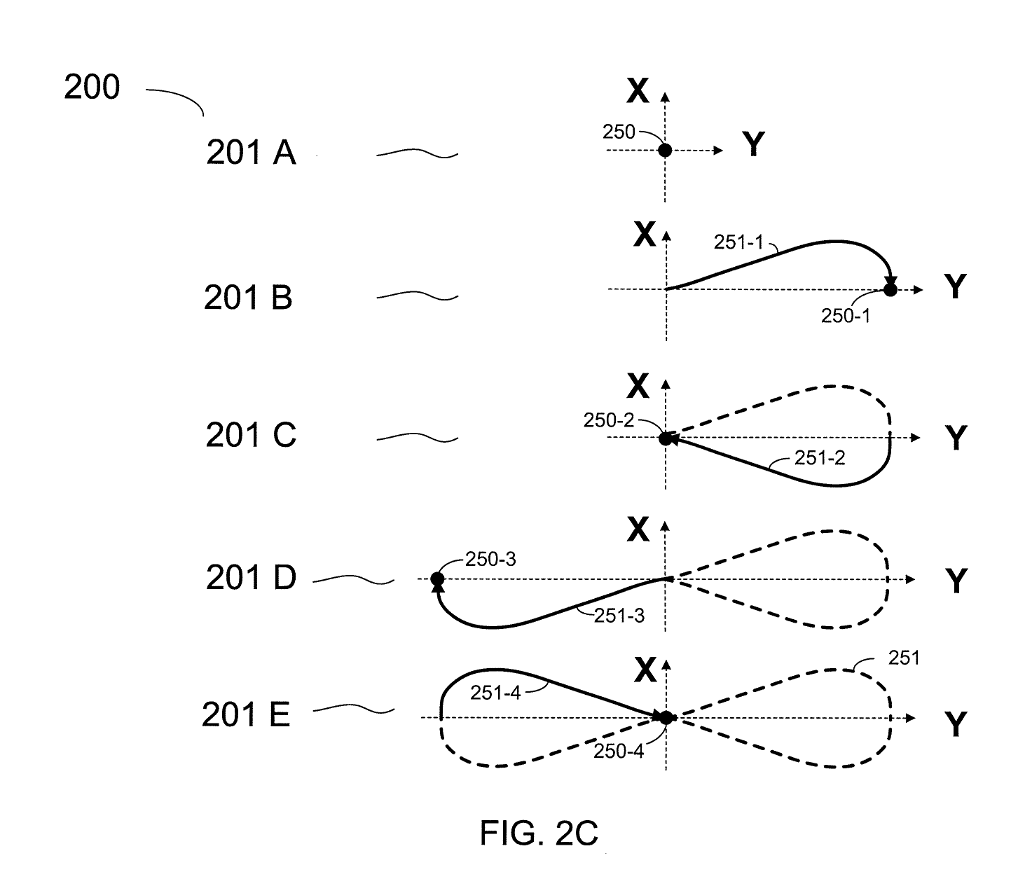

According to some embodiments consistent with FIGS. 1A and 1B, the length L₁ of hand-piece 150 is 3-4 inches (approximately 7.5 cm to 10 cm). The length L₂ of cannula assembly 110 is 30 mm. According to some embodiments, cannula assembly 110 may have a portion extending inside hand-piece 150, adding to the length L₂ shown in FIG. 1A. The length L₃ of the tapered portion of hand-piece 150 may depend on ergonomic and cosmetic considerations. In some embodiments length L₃ may be approximately 6 mm. Use of embodiments of endoprobe 100 for providing OCT scanning trajectories will be described in detail in relation to FIGS. 2A-2C below.

FIG. 2A illustrates a rotational mechanism 200 in a microsurgical endoprobe, according to some embodiments. A rotation or counter-rotation in embodiments consistent with the present disclosure is defined with respect to a reference point fixed in the surrounding of cannula assembly 110. For example, the reference point may be a point in the tissue surrounding cannula assembly 110. Further, in some embodiments a rotation or counter-rotation may be defined for either one of the proximal or distal elements (e.g. 160 and 170, see FIG. 1B) in reference to the other. An endoprobe used in embodiments consistent with the present disclosure may be such as endoprobe 100 above (see e.g., FIG. 1A). FIG. 2A includes a partial view of an endoprobe such as endoprobe 100 along a sagittal plane including the probe LA, proximal optical element 260, and distal optical element 270. An endoprobe as used in mechanism 200 may include a proximal optical element 260 and a distal optical element 270 forming a space or gap 265 between them, along the probe LA (see e.g., Z-axis in FIG. 2A). Proximal optical element 260 may be as proximal element 160 and distal optical element 270 may be as distal element 170, described in detail above, in relation to FIG. 1B. Embodiments consistent with the present disclosure may use optical elements as described in a paper by Wu et al. (J. Wu, M. Conry, C. Gu, F. Wang, Z. Yaqoob, and C. Yang; “'Paired-angle-rotation scanning optical coherence tomography forward-imaging probe” Optics Letters, 31(9) 1265 (2006)). Thus, in some embodiments consistent with the present disclosure a rotational motion is provided to proximal optical element 260 through inner tube 130. In some embodiments, a rotational motion is provided to distal optical element 270 through outer tube 140.

The gap between elements 260 and 270 may be limited by two angled faces of a lens on either side, in some embodiments. Rotational mechanism 200 may include configurations 201A through 201E as elements 260 and 270 are counter-rotated relative to each other, about the Z-axis (along the probe LA). In an X-Y plane perpendicular to the Z-axis in FIG. 2A and forming a right-handed coordinate system between coordinates X-Y-Z, distal element 270 may rotate ‘clockwise’ relative to an observer looking in the +Z direction, while proximal element 260 may rotate ‘counter-clockwise’ relative to the same observer. The starting configuration in rotational mechanism 200 may be such that elements 260 and 270 have their angled faces parallel to each other, such as configuration 201A. In configuration 201A, the gap between elements 260 and 270 has the same length across the X-Y plane. A 90° (degree) rotation of elements 260 and 270 produces configuration 201B. In configuration 201B, the gap between elements 260 and 270 is short along the +Y axis direction and long along the −Y axis direction. After a 180° rotation of elements 260 and 270 configuration 201C is obtained. In configuration 201C the gap between elements 260 and 270 has the same length across the X-Y plane. After a 270° rotation of elements 260 and 270 configuration 201D is obtained. In configuration 201D the gap between elements 260 and 270 is short along the −Y axis direction and long in the +Y direction. After a 360° rotation of elements 260 and 270 configuration 201E is obtained. Configuration 201E is substantially the same as configuration 201A, according to some embodiments.

A light beam 275 passing through elements 260 and 270 may be deflected from the probe LA in a direction forming an angle α relative to the probe LA, and an angle φ in the azymuthal direction. The value of angles α and φ depends on the configuration of the gap between elements 260 and 270 relative to the X-Y plane. This will be illustrated in detail below in relation to FIGS. 2B and 2C.

FIG. 2B illustrates rotational mechanism 200 in a microsurgical endoprobe, according to some embodiments. FIG. 2B includes a cross-sectional view of a rotational mechanism 200 for an endoprobe such as endoprobe 100, taken across the probe LA (+Z-axis). FIG. 2B illustrates the motion of a direction indicator 261 for proximal element 260 (see e.g., FIG. 2A) relative to the motion for a direction indicator 271 for distal element 270. Indicator 261 is an arrow pointing in the direction on the X-Y plane where an angled face in element 260 protrudes further along the +Z direction. Likewise, indicator 271 is an arrow pointing in the direction on the X-Y plane where an angled face in element 270 protrudes further along the −Z direction.

Thus, in embodiments consistent with rotational mechanism 200, configuration 201A (see e.g., FIG. 2A) may correspond to indicator 261 pointing in the −X direction, and indicator 271 pointing in the +X direction. Configuration 201B (see e.g., FIG. 2A) may correspond to indicators 261 and 271 pointing substantially in the same direction along the +Y direction. Note that according to rotational mechanism 200, proximal element 260 and distal element 270 rotate in opposite directions about the Z-axis. For example, in FIGS. 2A and 2B proximal element 260 rotates counter-clockwise looking into the +Z direction. Likewise, distal element 270 rotates clockwise looking into the +Z direction. The precise orientation of the rotation of elements 260 and 270 is not limiting. One of regular skill in the art would recognize embodiments with different configurations consistent with the present disclosure. Thus, in some embodiments of rotational mechanism 200 the azymuthal angle 265 formed between indicator 261 and the +X axis may increase. Likewise, in some embodiments of rotational mechanism 200 the azymuthal angle 275 formed between indicator 271 and the +X axis may be negative and increase in magnitude. Configuration 201C (see e.g., FIG. 2A) may correspond to indicator 261 pointing in the +X direction, and indicator 271 pointing in the −X direction. Configuration 201D (see e.g., FIG. 2A) may correspond to indicators 261 and 271 pointing substantially in the same direction along the −Y direction. Configuration 201E (see e.g., FIG. 2A) may correspond to indicator 261 pointing in the −X direction and indicator 271 pointing in the +X direction.

As elements 260 and 270 complete a full turn around the probe LA, the light beam completes a full sweep along a trajectory in the X-Y plane centered on the probe LA (Z-axis). This is described in detail in relation to FIG. 2C, below.

FIG. 2C illustrates a scanning trajectory 251 of an optical beam, according to some embodiments. In embodiments consistent with the present disclosure, an optical beam may traverse an endoprobe from proximal optical element 260 to distal optical element 270, travelling in a direction substantially parallel to the +Z axis (see e.g., FIG. 2A). After passing through proximal element 260 and distal element 270, an optical beam may be deflected by angles α and φ, as described above (see e.g., FIG. 2A) forming a spot 250 on tissue located in front of an endoprobe as disclosed herein. As optical elements 260 and 270 rotate according to rotational mechanism 200 (see e.g., FIGS. 2A and 2B) spot 250 forms trajectory 251 on an X-Y plane of the tissue.

Thus, optical information from a point 250 in the tissue located in front of an endoprobe as disclosed herein may be transmitted to a detector coupled to an analysis system located in the proximal side of the endoprobe. Further, optical elements 260 and 270 may collect optical information from a collection of spots 250 forming trajectory 251 as elements 260 and 270 rotate according to rotational mechanism 200 (see e.g., FIGS. 2A and 2B). The optical information may impinge distal element 270 from spot 250 coming at an angle α, and φ, as described above (see e.g., FIG. 2A).

For example, when the gap between elements 260 and 270 is constant across the X-Y plane, α may be substantially zero (0) and the light beam be substantially un-deflected. This may be the situation in configuration 201A and 201E, where spot 250 is located substantially on the Z-axis, at the origin of the X-Y plane (see e.g., FIG. 2A). When the gap between elements 260 and 270 is as in configuration 201B, α may have a maximum value and φ may be equal to −90°. Thus, a portion 251-1 of trajectory 251 is formed by spot 250 as rotational mechanism 200 turns configuration 201A into 201B, reaching spot 250-1. When the gap between elements 260 and 270 is as in configuration 201C, α may be substantially zero (0) and the light beam be substantially un-deflected. Thus, a portion 251-2 of trajectory 251 is formed by spot 250 as rotational mechanism 200 turns configuration 201B into 201C, reaching spot 250-2 close to the origin. In portion 251-2, spot 250 returns to a substantially un-deflected position close to the center of the X-Y plane. When the gap between elements 260 and 270 is as in configuration 201D, α may have a maximum value and φ may be equal to +90°. Thus, a portion 251-3 of trajectory 251 is formed by spot 250 as rotational mechanism 200 turns configuration 201C into 201D, reaching beam spot 250-3. When the gap between elements 260 and 270 is as in configuration 201E, α may be substantially zero (0) and the light beam be substantially un-deflected. Thus, a portion 251-4 of trajectory 251 is formed by spot 250 as rotational mechanism 200 turns configuration 201D into 201E, returning to beam spot 250-4.

Some embodiments consistent with the above description may use probe 100 in an OCT-scanning procedure. In this case, optical illumination takes place in the forward direction of the probe LA. In forward-directed scans, the target tissue may be ahead of the probe in the X-Y plane. Thus, light traveling from the tip of the probe to the tissue, and back from the tissue into the probe may travel in a direction substantially parallel to the probe LA. In some embodiments using forward-directed scans, the target tissue may be approximately on the X-Y plane, but not exactly. Furthermore, in some embodiments light traveling to and from the target tissue or from and into the probe may not be parallel to the probe LA, but form a symmetric pattern about the probe LA (Z axis).

OCT scanning procedures typically include an in-depth image obtained through an A-scan. For example, in embodiments consistent with the present disclosure an A-scan may be provided for a given beam spot 250 (see e.g., FIG. 2C). A collection of A-scans along a trajectory may form a two-dimensional (2-D) image in what is referred to as a B-scan. The two dimensions in an OCT B-scan as described above arise from combining a one-dimensional beam spot trajectory and a one-dimensional, in-depth, A-scan for each spot on the one-dimensional trajectory. In such cases, the two counter-rotating optical elements 260 and 270 may provide a B-scan of the light beam used in OCT imaging. For example, in embodiments consistent with the present disclosure a B-scan may be provided for trajectory 251. In some embodiments, a B-scan is provided by a combination of portions 251-1 through 251-4 in trajectory 251 (see e.g., FIG. 2C).

Performing repeated B-scans along different lines in the tissue, a 3-D rendition of the tissue may be provided in some embodiments consistent with the present disclosure. In some embodiments, the B-scans may be a set of lines having the same length and arranged in a radius from a common crossing point. Thus, the plurality of B-scans provides an image of a circular area in the tissue, having a depth. Such a scanning profile renders a 3-D image of the tissue known as a C-scan.

According to some embodiments of endoprobe 100 used for OCT-imaging, a plurality of A-scans may be completed for each B-scan step. For example, 512 A-scans may be used to complete one B-scan. Some embodiments may use a lower number of A-scans per B-scan cycle, thus allowing the B-scan procedure to take place at a faster rate. In such cases, the rotating and counter-rotating speeds of proximal element 260 and distal element 270 may be further increased.

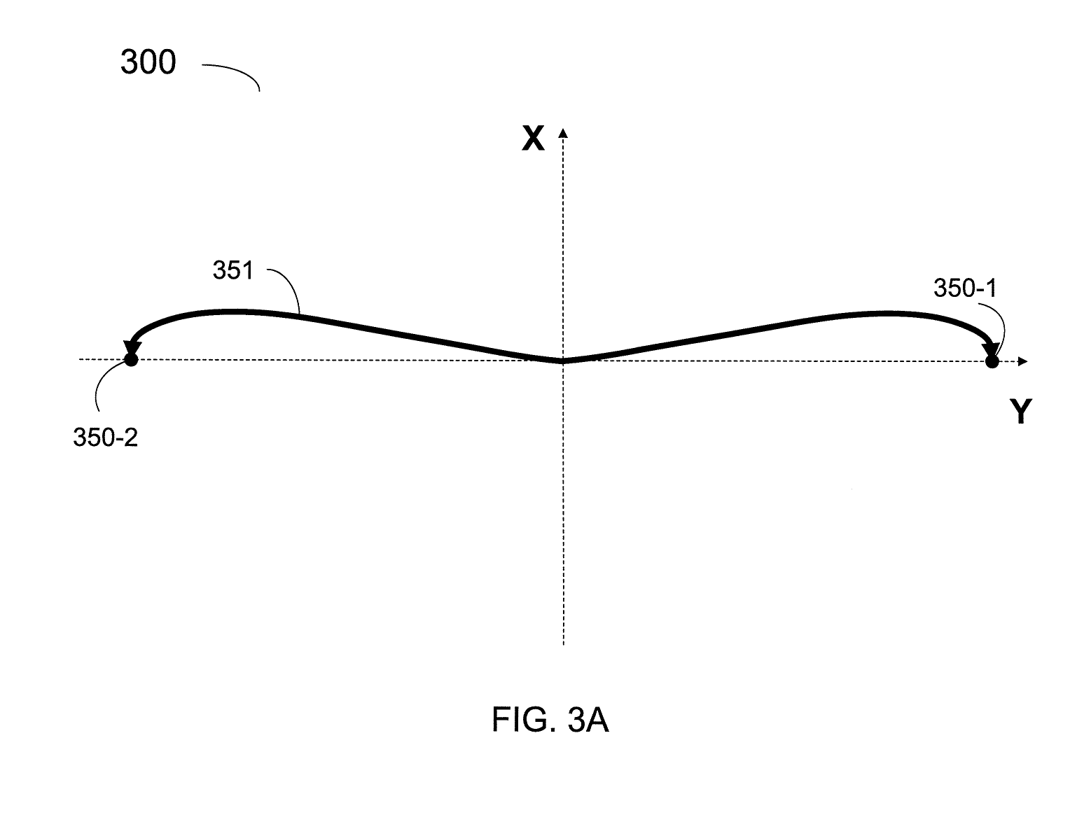

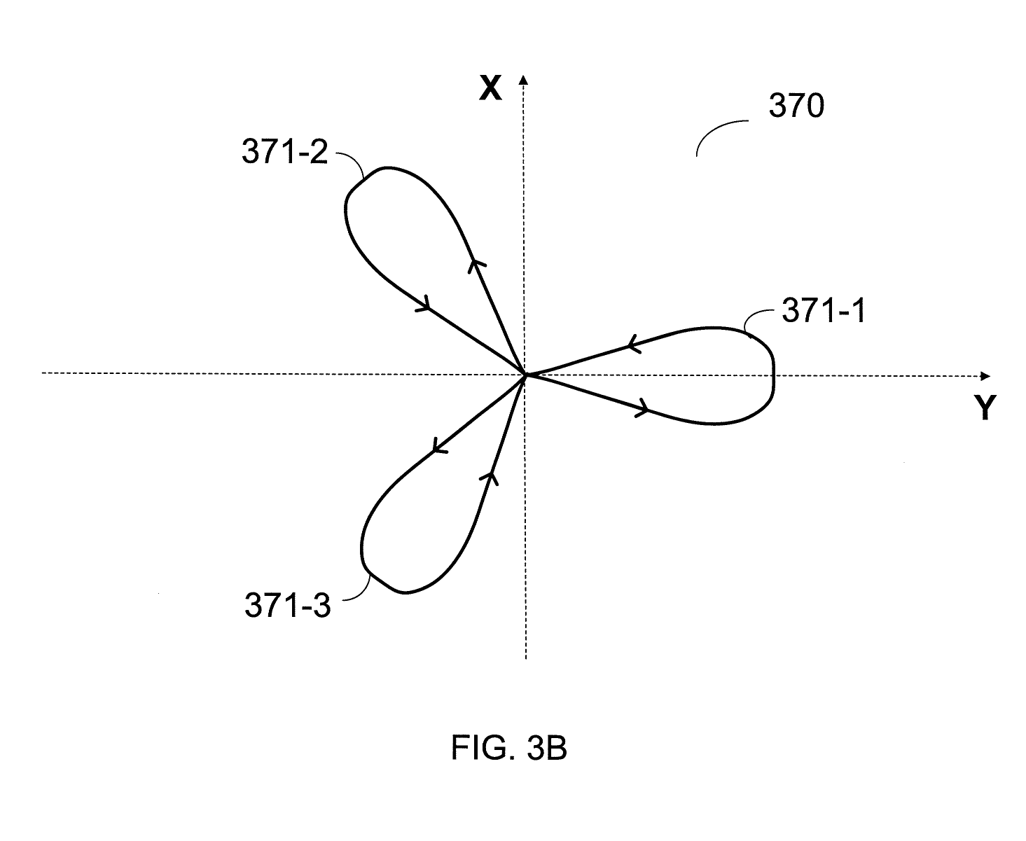

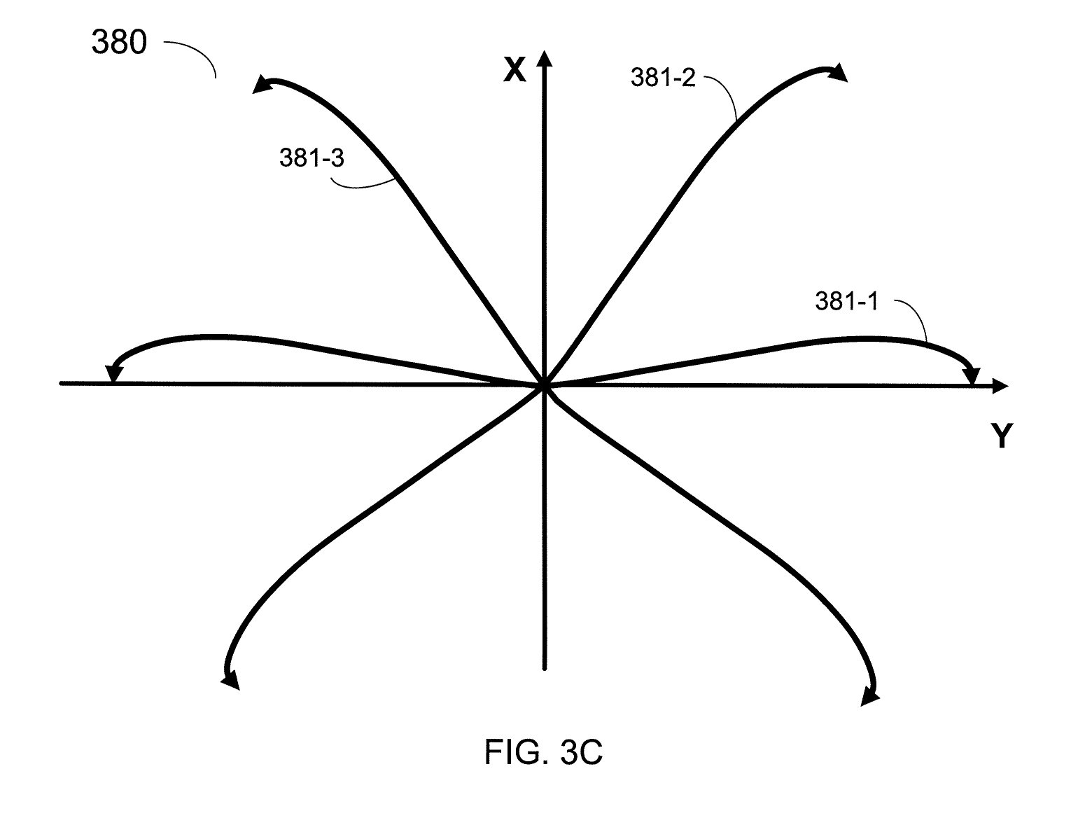

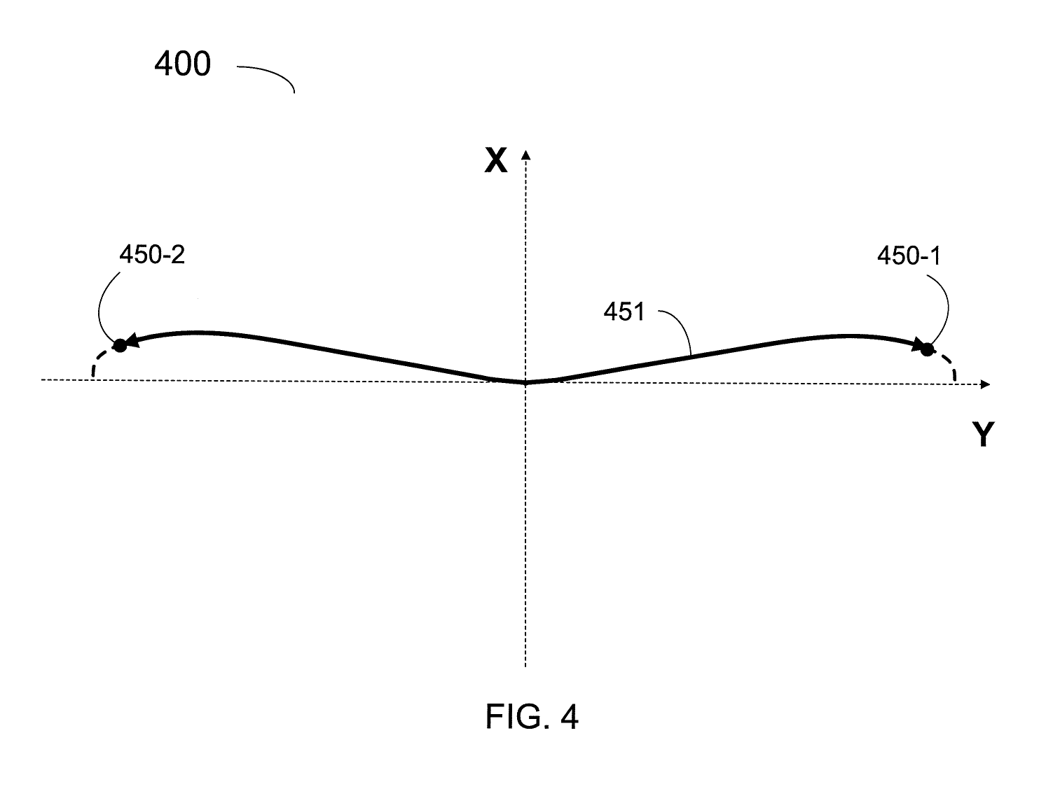

To obtain a complex set of scan lines, including B-scan lines arranged in pre-selected patterns, proximal element 260 and distal element 270 are precisely rotated with respect to one another. Control of the rotation of elements 260 and 270 is important for the efficacy of OCT procedures. In particular, repeatability of the motion may be required so that A-scans may be aligned along B-scan lines to conform a continuous image. In some embodiments, the rotation of elements 260 and 270 may include a periodic cycle. For example, a cycle may include a complete rotation of element 260 or 270 about the probe LA. In some embodiments, a cycle may include a partial rotation of element 260 or 270 about the probe LA. The resulting scanning trajectories from embodiments of rotational mechanisms consistent with the present disclosure will be described in detail with respect to FIGS. 3 and 4, below.

FIG. 3A illustrates a scanning trajectory 351 of an optical beam in rotational mechanism 300, according to some embodiments. Scanning trajectory 351 is a radial-oriented B-scan about the probe LA. For example, scanning trajectory 351 may be obtained by rotating proximal element 260 and distal element 270 continuously from configuration 201D, to configuration 201E, to configuration 201B. To achieve this, the rotation of proximal element 260 and distal element 270 may be synchronized, according to embodiments consistent with the present disclosure. In some embodiments, rotating proximal element 260 and distal element 270 synchronously includes moving element 260 and 270 in phase. Further, rotating proximal element 260 and distal element 270 synchronously may include rotating elements 260 and 270 at the same but opposite angular speed relative to the +Z-axis (see e.g., FIG. 2A). Trajectory 351 includes an edge spot 350-1 and an edge spot 350-2. In some embodiments, spots 350-1 and 350-2 define the length of trajectory 351. According to embodiments consistent with the present disclosure, spots 350-1 and 350-2 may be along the Y axis in the X-Y plane perpendicular to the probe LA. In some embodiments, spots 350-1 and 350-2 may be along any direction on the X-Y plane perpendicular to the probe LA, defining an angle of approximately 180° having a vertex at the origin of the X-Y plane.

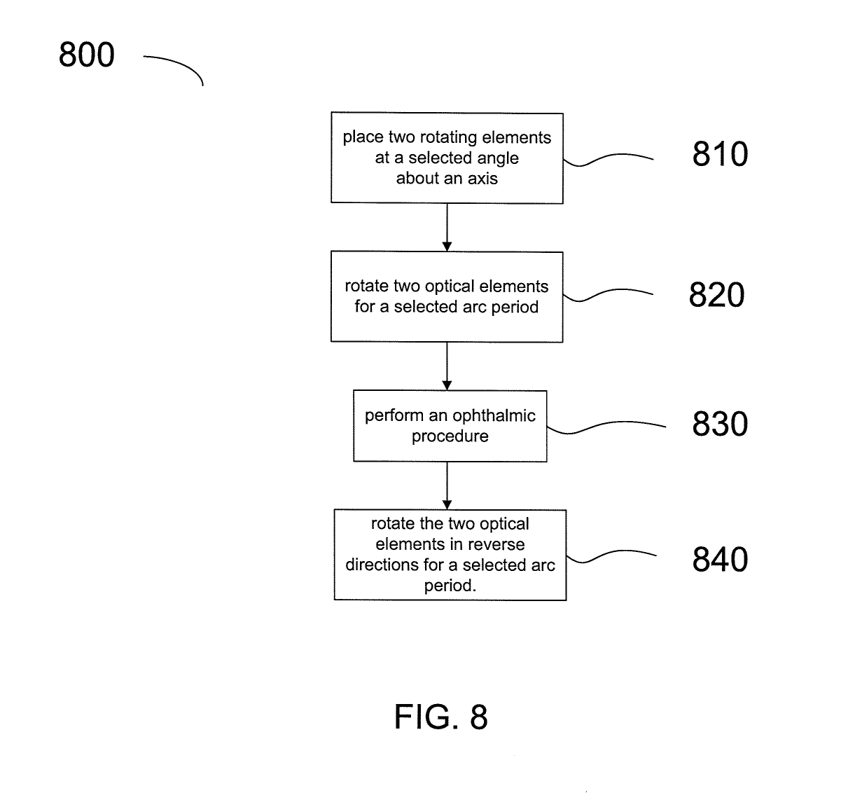

In embodiments such as disclosed herein each of proximal element 260 and distal element 270 rotates in either direction by a total of 180°. For example, in rotational mechanism 200 the transition from configuration 201D to configuration 201E, to configuration 201B includes a 180° clockwise rotation of distal element 270 and a 180° counter-clockwise rotation of proximal element 260 (see e.g., FIG. 2A). Thus, a beam spot may follow trajectory 351 from spot 350-1 to spot 350-2. By reversing the rotation of proximal element 260 and distal element 270 the opposite scan trajectory is obtained. Thus, by rotating proximal element 260 clockwise about the +Z axis by 180° and distal element 270 counter-clockwise about the +Z-axis by 180°, a beam spot may follow trajectory 351 from spot 350-2 to spot 350-1. If both proximal element 260 and distal element 270 were to rotate by 360° in opposite directions, the resulting trajectory would by a FIG. 8, with a mirror image of trajectory 351 about the Y-axis completing the loop (see e.g., FIG. 2C, element 201E).

According to some embodiments consistent with the present disclosure, a rotational mechanism may provide a reciprocating motion to proximal element 260 and distal element 270. For example, a reciprocating motion may include a first cycle where proximal element 260 and distal element 270 transition from configuration 201B, to configuration 201C, to configuration 201D (see e.g., FIG. 2A). In the first cycle, proximal element 260 may rotate counter-clockwise and distal element 270 may rotate clockwise relative to the +Z axis. A reciprocating motion may also include a second cycle where the rotation of proximal element 260 and distal element 270 is reversed, transitioning back from configuration 201D to configuration 201C, to configuration 201B (see e.g., FIG. 2A). In the second cycle, proximal element 260 may rotate clockwise and distal element 270 may rotate counter-clockwise relative to the +Z axis.

The orientation of trajectory 351 in the X-Y plane may be adjusted by choosing the relative rotation phase between elements 260 and 270. For example, according to some embodiments, spot 350-1, having a maximum +Y coordinate in trajectory 351 corresponds to configuration 201B (see e.g., FIG. 2A). According to FIG. 2A, in configuration 201B the gap between elements 260 and 270 has a minimal length along the +Y direction. Likewise, spot 350-2 having a minimum −Y coordinate in trajectory 351 corresponds to configuration 201D (see e.g., FIG. 2A). Configuration 201D is similar to configuration 201B, rotated by 180° about the Z-axis. According to FIG. 2A, in configuration 201B the gap between elements 260 and 270 has a minimal length along the +Y direction. Also according to FIG. 2A, in configuration 201D the gap between elements 260 and 270 has a minimal length along the −Y direction. The phase between the rotation of elements 260 and 270 is adjusted by using a configuration having the minimum gap distance between elements 260 and 270 oriented in any desired direction φᵣₒₜ along the X-Y plane. Thus, a reciprocating motion by 180° of elements 260 and 270 will result in a trajectory such as 351 rotated by an angle φᵣₒₜ about the origin in the XY plane (see e.g., FIG. 3A).

According to embodiments disclosed herein, trajectory 351 may have a 2-D profile in the X-Y plane, having a length defined by the distance between spots 350-1 and 350-2, and a width (see e.g., FIGS. 2C and 3). The width of trajectory 351, is the maximum +X coordinate value attained by spots in trajectory 351 (see e.g., FIG. 3A). In some embodiments, the width may be much smaller than the length of trajectory 351. Furthermore, in embodiments of the present disclosure used for OCT scanning, the width of trajectory 351 may be negligible compared to the spot size of an optical beam scanned across the tissue. Thus, in embodiments consistent with the present disclosure a trajectory such as trajectory 351 may be substantially a straight line along the X-Y plane. In such embodiments, a proximal element 260 rotates at a speed dφ₁ substantially equal to a counter-rotating speed dφ₂ of distal element 270. In embodiments consistent with this disclosure used for OCT scanning, a B-scan with a trajectory such as 351 is part of a plane including the probe LA.

FIG. 3B illustrates a scanning trajectory 370 of an optical beam, according to some embodiments. By adjusting the relative angular speeds and phases of proximal element 260 and distal element 270 the B-scan plane may be rotated about the probe LA (+Z axis in FIGS. 2A-2C). Thus, some embodiments consistent with the present disclosure used for OCT scanning may provide a C-scan, such as scanning trajectory 370. For example, a rotational mechanism consistent with the present disclosure may have proximal element 260 rotating at a speed dφ₁ greater than a speed dφ₂ of a counter-rotating distal element 270. In such configuration a flower pattern such as 370, having three lobes 371-1, 371-2, and 371-3, is formed. Note that in FIG. 3B scanning trajectory follows generally a counter-clockwise rotation. The specific direction of scanning trajectory 370 depends on the orientation of the rotation of elements 260 and 270, and which of the speeds dφ₁ and dφ₂ is greater than the other. The number of lobes in scanning trajectory 370 is not limiting and depends on the relative difference between rotating and counter-rotating speeds dφ₁ and dφ₂. For example, the number of lobes in scanning trajectory 370 increases as the difference between rotational and counter-rotational speeds dφ₁ and dφ₂ grows.

FIG. 3C illustrates a scanning trajectory 380 of an optical beam, according to some embodiments. Scanning trajectory 380 is a rotating line including linear scans 381-1, 381-2, and 381-3. In embodiments consistent with the present disclosure, scanning trajectory 380 may be obtained using dφ₁=dφ₂. For each line scan 381-1, 382-2, and 381-3 a fixed phase difference between the rotation and counter-rotation of elements 260 and 270 is used. The fixed phase difference is changed after each line scan 381-1, 381-2, and 381-3 is completed. This can be achieved by stopping or slowing one of the scanning elements 260 or 270 for a fraction of a rotation.

FIG. 4 illustrates a scanning trajectory 451 of an optical beam, according to some embodiments. Trajectory 451 includes an edge spot 450-1 and an edge spot 450-2. According to embodiments consistent with the present disclosure, the length of trajectory 451 is mostly determined by the distance between spots 450-1 and 450-2. Scanning trajectory 451 is similar to trajectory 351 (see e.g., FIG. 3A). In some embodiments, scanning trajectory 451 is obtained by a rotational mechanism 400 similar to mechanism 200, as described in detail above in relation to trajectory 351. In some embodiments, edge spots 450-1 and 450-2 may be closer to one another than edge spots 350-1 and 350-2 are (see e.g., FIG. 3A). Thus, the rotational mechanism moving proximal element 260 and distal element 270 may provide a spot moving along trajectory 451 from left to right, stopping at spot 450-1 before the maximum +Y spot available (350-1, see e.g., FIG. 3A). Likewise, a rotational mechanism consistent with the present disclosure may reverse the scanning trajectory from spot 450-1 and move along trajectory 451, to the left, stopping at spot 450-2 before the minimum −Y spot available (350-2, see e.g., FIG. 3A).

Some embodiments of a rotational mechanism consistent with the present disclosure may provide trajectory 451 by a reciprocating motion between proximal element 260 and distal element 270. For example, a rotational mechanism may provide trajectory 451 by moving elements 260 and 270 from a configuration 201B′ which is between configuration 201B and 201C (see e.g., FIG. 2A). A rotational mechanism then transitions from configuration 201B′ to configuration 201C, to configuration 201C′. Configuration 201C′ may be a configuration between 201C and configuration 201D.

Thus, according to embodiments consistent with the present disclosure a trajectory 451 may be obtained by a rotational mechanism providing a reciprocating motion to proximal element 260 and distal element 270. The reciprocating motion includes a first cycle where element 260 rotates clockwise for an angle φ₁ less than 180°. Further, in some embodiments during the first cycle element 270 rotates counter-clockwise for an angle φ₁, synchronously with element 260. In a second cycle, the reciprocating motion may rotate element 260 counter-clockwise for an angle φ₂ less than 180°. In some embodiments, during the second cycle element 270 rotates clockwise for an angle φ₂, synchronously with element 260. In some embodiments, angles φ₁ and φ₂ are equal.

In some embodiments, the orientation of trajectory 451 in the X-Y plane may be adjusted by choosing the relative phase between the rotation of proximal element 260 and distal element 270, as discussed in detail above with respect to trajectory 351. Also, some embodiments used in OCT scanning may provide a C-scan by rotating trajectory 451 about the origin in the X-Y plane, as described in detail above with respect to trajectory 351.

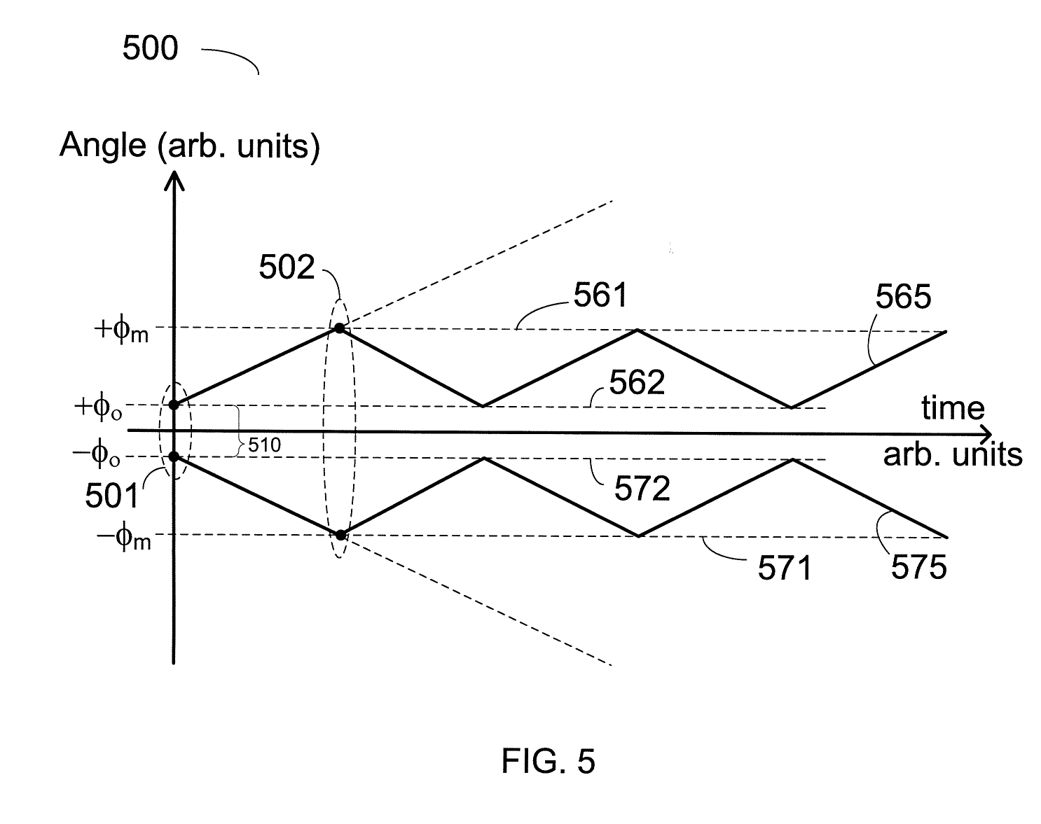

FIG. 5 illustrates a chart 500 for a rotational mechanism in a microsurgical endoprobe, according to some embodiments. A rotational mechanism according to some embodiments is rotational mechanism 200 using proximal optical element 260 and distal optical element 270 (see e.g., FIGS. 2A-2C). Furthermore, in embodiments consistent with the present disclosure a rotational mechanism may use endoprobe 100 having elements 160 and 170 as proximal optical element 260 and distal optical element 270, respectively (see e.g., FIG. 1B). Chart 500 depicts time in the abscissas, and angle in the ordinates. According to some embodiments, the ordinate of FIG. 5 depicts azymuthal angle φ, measured counter-clockwise relative to the +X-axis in an X-Y plane perpendicular to a probe LA (see e.g., FIG. 2A). Chart 500 includes curve 565 showing the time dependence of an angular rotation of a proximal element, such as proximal optical element 260 (see e.g., FIG. 2A). Chart 500 also includes curve 575 showing the time dependence of an angular rotation of a distal element such as distal optical element 270 (see e.g., FIG. 2A). Curves 575 and 565 are separated at an initial time by a gap 510, indicating a first configuration 501. First configuration 501 may be a configuration such as 201A-201D in FIG. 2A. First configuration 501 includes an initial orientation of a proximal element 562, and an initial orientation of a distal element 572. Gap 510 is the difference between the initial orientation of a proximal element 562 and the initial orientation of a distal element 572.

FIG. 5 shows initial orientations 562 and 572 having the same magnitude and opposite value (±φ₀). Some embodiments may have orientations 562 and 572 having different values. For example, for rotational mechanism 200 with first configuration 501 as configuration 201A, gap 510 may be equal to 180°. In such case, initial orientation 562 is 180° and initial orientation 572 is zero (0). The precise value of angles depicted in chart 500 is dependent on the choice of reference axis. Thus, the precise value of angles depicted in chart 500 is not limiting. One of regular skill in the art would recognize that the abscissas axis in chart 500 may be located at any height along the ordinates, without loss of generality.

Chart 500 includes a first cycle where a rotational mechanism turns an endoprobe as disclosed herein from first configuration 501 to a second configuration 502. In the first cycle, curve 565 increases at a constant angular speed dφ₅₆₁ while curve 575 decreases at a constant speed dφ₅₇₁. In some embodiments consistent with the present disclosure dφ₅₆₁=dφ₅₇₁. One of regular skill in the art may recognize that dφ₅₆₁ may be different from dφ₅₇₁. In second configuration 502, curve 565 reaches a maximum value 561 (φₘ), and curve 575 reaches a minimum value 571 (−φₘ). The magnitude of value 561 may be the same as that of value 571 (such as φₘ, see e.g., FIG. 5), according to some embodiments. In some embodiments the magnitude of value 561 may be different from the magnitude of value 571. Further according to some embodiments, the difference between maximum angle 561 and initial orientation 562 may be less than or equal to 180°. In some embodiments, the difference between initial orientation 572 and minimum angle 571 may be less than or equal to 180°. Further according to some embodiments, the difference between initial orientation 572 and minimum angle 571 may be the same or approximately the same as the difference between maximum angle 572 and initial orientation 562.

Chart 500 includes a second cycle where a rotational mechanism turns an endoprobe as disclosed here from second configuration 502 to first configuration 501. According to embodiments consistent with the present disclosure, a second cycle may include reversing the rotation direction of a proximal element and a distal element in relation to the first cycle. In the second cycle curve 565 decreases at a constant angular speed ddφ₅₆₁ while curve 575 increases at a constant speed ddφ₅₇₁. In some embodiments consistent with the present disclosure ddφ₅₆₁=ddφ₅₇₁. One of regular skill in the art may recognize that ddφ₅₆₁ may be different from ddφ₅₇₁. In the second cycle, curve 565 decreases to value 562 (φₒ), and curve 575 increases to value 572 (−φₒ). In some embodiments consistent with the present disclosure, the values for d₅₆₁ and dd₅₆₁ may be substantially equal, and the values for d₅₇₁ and dd₅₇₁ may be substantially equal. In some embodiments, d₅₆₁ may be different from dd₅₆₁, and d₅₇₁ may be different from dd₅₇₁.

A rotational mechanism as disclosed herein provides a reciprocating motion to an endoprobe between first configuration 501 and second configuration 502. The rotational mechanism includes a first cycle turning an endoprobe from first configuration 501 to second configuration 502. The rotational mechanism also includes a second cycle turning the endoprobe from second configuration 502 to first configuration 501. The second cycle includes reversing the direction of rotation of a proximal element and a distal element in an endoprobe as disclosed herein, relative to the first cycle. Embodiments of the present disclosure may use inner tube 130 to provide an angular rotation following curve 565 to proximal element 160 (see e.g., FIG. 1B). Also, embodiments of the present disclosure may use outer tube 140 to provide an angular rotation to distal element 170 according to curve 575. A reciprocating motion to outer tube 140 according to curve 575 avoids winding of vitreous humor during ophthalmic procedures when outer tube 140 is in direct contact with ocular tissue or vitreous humor. This allows the incorporation of virtually the entire cross-section of cannula assembly 110 for the purpose of optical collection (see e.g., FIG. 1B).

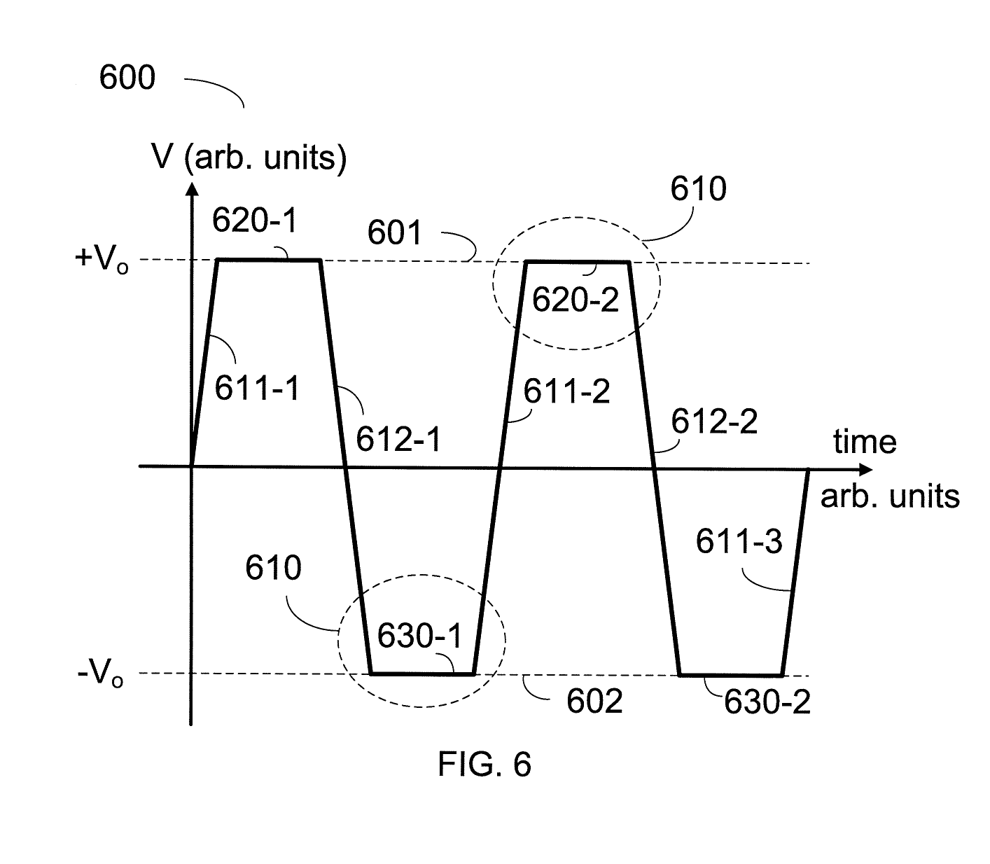

In some embodiments, the diameter of optical beam 275 is about 50% or more of the cannula assembly cross section (D₂, in FIG. 1A). In some embodiments, the diameter of optical beam 275 is 60% or more of D₂. Further according to some embodiments, the diameter of optical beam 275 is about 70% or more of D₂ (see e.g., Table I). According to some embodiments, the diameter of optical beam 275 is determined by the diameter of optical elements 260 and 270. For example, in some embodiments the diameter of optical beam 275 is equal to or approximately equal to the diameter of distal optical element 270. The SNR of an OCT system using endoprobe 100 according to methods disclosed herein is thus enhanced. Some embodiments consistent with the present disclosure provide a rotational speed to a proximal element or a distal element such as described in detail below, in relation to FIG. 6.

FIG. 6 illustrates a chart 600 for a rotational mechanism in a microsurgical endoprobe, according to some embodiments. Chart 600 depicts time in the abscissas, and rotational speed (V) in the ordinates. According to embodiments disclosed herein, positive V refers to counter-clockwise angular motion, and negative V refers to clockwise angular motion. For example, in embodiments consistent with the present disclosure clockwise and counter-clockwise orientations are defined relative to a +Z axis oriented along a probe LA (see e.g., FIGS. 2A-2C). Chart 600 includes curve 610 illustrating the rotational speed provided to a proximal element or a distal element in a rotational mechanism consistent with the present disclosure. Curve 610 includes ramp-up portions 611-1 through 611-3In ramp-up portions the rotational speed increases from either zero (0) as in portion 611-1, or from a negative value 602 (−Vo) as in portions 611-2 and 611-3, to a positive value 601 (+Vo). Curve 610 includes counter-clockwise portions 620-1 and 620-2, during which the rotational speed of a proximal element or a distal element is kept constant at a positive value 601 (+Vo). Curve 610 includes ramp-down portions 612-1 and 612-2. In ramp-down portions 612-1 and 612-2 the rotational speed decreases from a positive value 601 (+Vo) to a negative value 602 (−Vo). Curve 610 also includes clockwise portions 630-1 and 630-2, during which the rotational speed of a proximal element or a distal element is kept constant at negative value 602 (−Vo).

In embodiments consistent with the present disclosure, a first cycle in a rotational mechanism as described above in relation to FIG. 5 may include portions 611-1 and 620-1 in chart 600. The first cycle may further include part of portion 612-1 having positive rotational speed. Also according to embodiments disclosed herein, a second cycle in a rotational mechanism as described above in relation to FIG. 5 may include portion 630-1. The second cycle may also include parts of portions 612-1 and 611-2 having negative rotational speed.

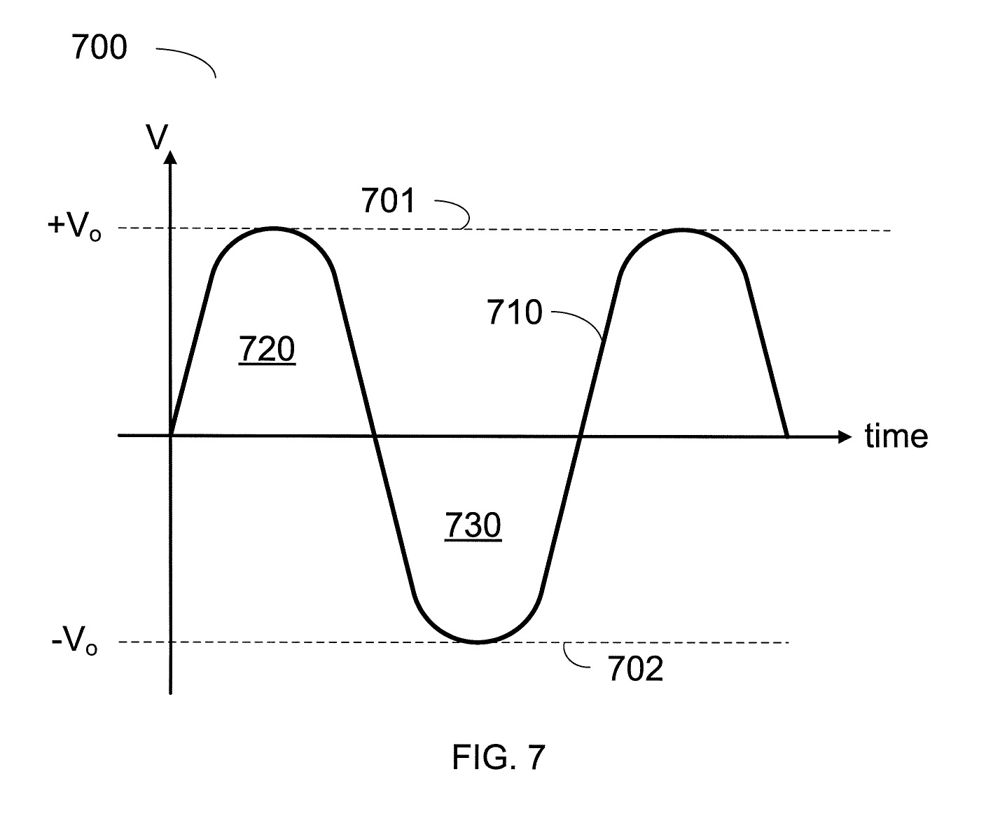

In embodiments consistent with the present disclosure, the magnitude of counter-clockwise rotational speed 601 may be substantially the same as the magnitude of clockwise rotational speed 602, namely Vo. In some embodiments, the magnitude of rotational speed 601 may be different from the magnitude of rotational speed 602. Further according to some embodiments, ramp-up portions 611-1 through 611-3 and ramp-down portions 612-1 and 612-2 have a high slope. Thus, during portions 611-1 through 611-3, 612-1, and 612-2, a proximal element or a distal element may be accelerated fast in order to attain a constant rotational speed 601 or 602. Some embodiments of a rotational mechanism as disclosed herein may implement a smooth rotational speed transition from a positive value to a negative value. This is illustrated in detail in relation to FIG. 7, below. Further, embodiments consistent with the present disclosure may be such that value 601 is as d₅₆₁, and value 602 has the same magnitude as d₅₇₁, as described in detail above in conjunction with FIG. 5.

FIG. 7 illustrates a chart 700 for a rotational mechanism in a microsurgical endoprobe, according to some embodiments. Chart 700 depicts time in the abscissas, and rotational speed (V) in the ordinates. According to embodiments disclosed herein, positive V refers to counter-clockwise angular motion, and negative V refers to clockwise angular motion. For example, in embodiments consistent with the present disclosure clockwise and counter-clockwise orientations are defined relative to a +Z axis oriented along a probe LA (see e.g., FIGS. 2A-2C). Chart 700 includes curve 710 illustrating the rotational speed provided to a proximal element or a distal element in a rotational mechanism consistent with the present disclosure. According to some embodiments, a rotational speed in curve 710 may have an acceleration substantially different from zero (0) during most of the time. Further according to some embodiments, curve 710 may have a sinusoidal profile in order to smooth out changes in a rotational speed provided to a proximal element or a distal element. According to curve 710, a proximal element or a distal element in an endoprobe consistent with the present disclosure may transition smoothly from a positive rotational speed 701 (+Vo) to a negative rotational speed 702 (−Vo).

Embodiments consistent with the present disclosure avoid backlash effects in an endoprobe driven by a rotational mechanism as illustrated in FIG. 7. Other effects avoided by a rotational mechanism as in curve 710 are discontinuities in a scanning trajectory due to finite changes in acceleration of the rotational speed. A finite change in acceleration may result in a ‘jump’ or ‘jerk’ of the mechanical elements in an endoprobe, such as proximal element 160 and distal element 170 in endoprobe 100 (see e.g., FIG. 1B). This may result in a discontinuity in a scanning trajectory such as trajectory 351 or 451 (see e.g., FIGS. 3 and 4, above). In embodiments used for OCT scanning it may be desirable to avoid discontinuities in the scanning trajectory, as these may be difficult to fix by software.

In embodiments of a rotational mechanism consistent with chart 700 used for OCT, a correction factor may be used during data collection. The correction factor accounts for the nonlinear displacement of beam spot 250 along scanning trajectory 251, when the rotational speed of proximal element 260 and distal element 270 follows curve 710 (see e.g., FIGS. 2A-2C, and 7). For example, in embodiments such as disclosed herein used in OCT scanning, an A-scan may be provided for beam spots 250 evenly distributed in time. In this configuration, a correction factor accounts for the nonlinear change in position of beam spot 250 during even time intervals in a rotational motion, as depicted in curve 710. Thus, a B-scan is appropriately assembled from a collection of A-scans along trajectory 251. In some embodiments used in OCT scanning, an A-scan may be provided during selected time intervals such that the spots 250 for which the A-scan is collected are evenly distributed in space, along trajectory 251.

Curve 710 includes positive portion 720 and negative portion 730, according to some embodiments. Portion 720 includes points having a positive rotational speed, and portion 730 includes points having a negative rotational speed. In embodiments consistent with the present disclosure, a first cycle in a rotational mechanism as described above in relation to FIG. 5 may include portion 720. Also according to embodiments disclosed herein, a second cycle in a rotational mechanism as described above in relation to FIG. 5 may include portion 730.

FIG. 8 illustrates a flow diagram for a method 800 to implement a rotational mechanism for a microsurgical endoprobe, according to some embodiments. Method 800 may be used in conjunction with endoprobe 100 as disclosed herein (see e.g., FIGS. 1A and 1B). Method 800 may be performed automatically by a computer in a remote console or controller device coupled to an endoprobe as disclosed herein. In some embodiments, method 800 may be performed manually by an operator handling endoprobe 100. Further according to some embodiments, method 800 may be performed partially by a computer in the remote console and by an operator handling endoprobe 100.

Method 800 includes 810 for placing two rotating elements at a selected angle about an axis. In some embodiments, two rotating elements in 810 may be inner tube 130 and outer tube 140 as in an endoprobe 100 (see e.g., FIG. 1). At 820 two optical elements rotate for a selected arc period. At 820, the two optical elements are proximal optical element 260 and distal optical element 270, according to embodiments disclosed herein (see e.g., FIG. 2A). In embodiments consistent with the present disclosure the two optical elements in 820 may be proximal optical element 260 attached to inner tube 130, and distal optical element 270 attached to outer tube 140. At 830, an ophthalmic procedure may be performed. According to some embodiments, 830 may be performed in conjunction with 820. For example, in some embodiments after a rotation for a small arc period an endoprobe is stopped and an ophthalmic procedure is performed as in 830. Then, a rotation for a small arc period is performed as in 820 before stopping at a new location for a new ophthalmic procedure. In some embodiments, 820 and 830 may be performed simultaneously and independently. For example, as the two optical elements are rotated as in rotational mechanism 200, ophthalmic procedures may be performed on the tissue surrounding an endoprobe.

An ophthalmic procedure according to embodiments used for OCT may include collecting an A-scan at a spot 250 in a scanning trajectory 251 (see e.g., FIG. 2A). In some embodiments, an ophthalmic procedure in 830 may include use of a laser beam for therapeutic purposes. For example, in photodynamic procedures a laser light activates a chemical agent present in a drug previously delivered to the target tissue. In some embodiments, 830 may include use of laser light to oblate or remove tissue and residual materials from the areas of interest. An ophthalmic procedure in 830 may include distributing therapeutic laser pulses across the retina. This may be the case for procedures such as pan retinal photocoagulation.

Method 800 may also include 840 for rotating the two optical elements in reverse directions for a selected arc period. Thus, in embodiments consistent with the present disclosure beam spot 250 may traverse trajectory 251 in the opposite direction relative to 820, during 840 (see e.g., FIG. 2A). Further according to embodiments consistent with the present disclosure, 840 is performed in conjunction with 830. Thus, as beam spot 250 traverses trajectory 251 in the opposite direction relative to 820, an ophthalmic procedure as described above is performed at regular intervals.

According to embodiments consistent with the present disclosure, 830 may be performed in conjunction with 820 for a selected first set of spots along a scanning trajectory. Further, 830 may be performed in conjunction with 840 for a selected second set of spots along a scanning trajectory. For example, the first and second sets of spots may be spots 250 in trajectory 251 according to rotational mechanism 200 (see e.g., FIGS. 2A-2C). In some embodiments, the first set of spots may not include any spot in the second set of spots. Furthermore, in some embodiments the first set of spots and the second set of spots are selected to be evenly distributed along a scanning trajectory. A scanning trajectory consistent with method 800 as disclosed herein may be as trajectory 251 (see e.g., FIG. 2C), trajectory 351 (see e.g., FIG. 3A), or trajectory 451 (see e.g., FIG. 4). Thus, in some embodiments the first set of spots is intercalated with the second set of spots along a scanning trajectory.

In some embodiments, a first set of spots may be collected during a first cycle as described in relation to FIG. 6. Thus, the first set of spots may include spots collected during portion 611-1, counter-clockwise portion 620-1, and the positive part of portion 612-1 (see e.g., FIG. 6). Likewise, a second set of spots may include spots collected during a second cycle as described in relation to FIG. 6. Thus, the second set of spots may include spots collected during the negative part of portion 612-1, clockwise portion 630-1, and the negative part of portion 611-2. Embodiments consistent with this configuration may not use data collected from spots selected during accelerating and decelerating portions 611-1, 611-2, and 612-1. In some embodiments, data collected during accelerating and decelerating portions 611-1, 611-2, and 612-1 may be used by considering a correction factor as described above.

Some embodiments may include a first set of spots collected during counter-clockwise portion 620-1 only (see e.g., FIG. 6). Likewise, a second set of spots may include spots collected during clockwise portion 630-1 only (see e.g., FIG. 6). A configuration such as the above avoids collection of spots in portions of curve 610 where the rotational velocity is not constant (see e.g., FIG. 6). Thus, a correction factor such as described above in relation to curve 710 (see e.g., FIG. 7) may not be used in some embodiments. Furthermore, according to some embodiments consistent with the present disclosure, curve 610 is selected such that the arc period covered during portions 620-1, 620-2, 630-1, and 630-2 has a magnitude of about 180°. In such configurations, proximal and distal elements may move further than 180° (including acceleration and deceleration portions 611 and 612). In such embodiments data is collected during the 180° rotation of proximal and distal elements in portions 620 and 630 only (e.g., trajectory 351 is collected, see FIG. 3A). In some embodiments only data from portions 620 and 630 is collected, and portions 620 and 630 cover an arc period smaller than 180°. Thus, spots from a trajectory such as 451 are collected (see e.g., FIG. 4).

In some embodiments consistent with this disclosure, 830 may be performed in conjunction with 820, and not during 840. For example, in embodiments used for OCT scanning, data may be collected only during a first cycle of motion (see e.g., FIG. 6), such as counter-clockwise portions 620-1 and 620-2, only. Such embodiments may avoid having to correct for backlash in the mechanical parts of an endoprobe during reversal of the rotational mechanism. Without loss of generality, some embodiments may avoid backlash by collecting data during clockwise portions 630-1 and 630-2, only.

Embodiments consistent with the present disclosure may enable the use of the maximum possible scan range given by a specific design of cannula assembly such as cannula 110 (see e.g., FIG. 1A and 1B). This may be obtained when the arc period covered during rotation of proximal and distal elements is, in some embodiments, at least 180°. Some embodiments may use a shorter scan range, e.g., a smaller arc period rotation such as 90° or smaller, to provide a faster scanning configuration. Some embodiments may use a shorter scan range to reduce the damage or stress to tissue or vitreous humor surrounding the cannula assembly. Some embodiments may use a shorter scan range to use a portion of the scanning trajectory closer to a straight line, such as the central portion in trajectory 451 (see e.g., FIG. 4).

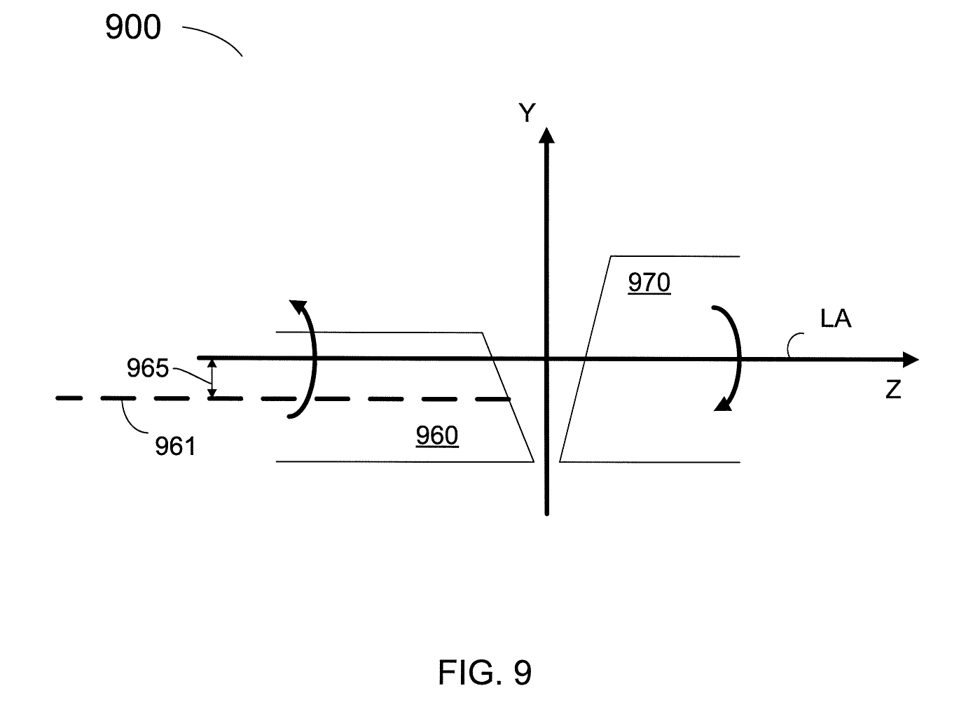

FIG. 9 illustrates an eccentric configuration 900 for a rotational mechanism in a microsurgical endoprobe, according to some embodiments. In FIG. 9, a proximal element 960 is placed with its optical axis 961 shifted by an offset 965 from the LA of an endoprobe. Distal element 970 is placed concentric to the LA of the endoprobe, that is, the optical axis of distal element 970 is collinear with the LA. Proximal element 960 and distal element 970 may be as proximal element 260 and distal element 270 described in detail above (see e.g., FIG. 2A). In embodiments consistent with the present disclosure, elements 960 and 970 are rotated and counter-rotated about the LA of the endoprobe. Thus, proximal element 960 translates along a circular trajectory, or a portion of it, in a rotational mechanism according to the present disclosure.

Proximal optical element 960 may be attached to inner tube 130 and a distal optical element 970 attached to outer tube 140, such that proximal optical element 960 has an optical axis 961 slightly off the mechanical axis of inner tube 130. In such configuration, a trajectory of an optical beam makes a loop travelling along an upper portion in one half of the trajectory and completing a lower portion in the second half of the trajectory. Such configuration essentially doubles the speed of a volume acquisition during a C-scan. Also, some embodiments may use an eccentric arrangement of elements 960 and 970 to provide a spatial dual scan differentiation. For example, surface gradient measurements may be obtained using a slightly eccentric configuration for elements 960 and 970. The eccentricity of proximal and distal optical elements may be chosen appropriately for different applications. For example, some applications having proximal element 960 and distal optical element 970 with diameters between about 0.5 mm to about 1 mm use an offset 965 of about 100 μm (=0.1 mm) from the mechanical axis of assembly 110, such as the LA of endoprobe 100. Embodiments consistent with the present disclosure may introduce an eccentricity into the configuration of elements 960 and 970 by mechanically placing proximal element 960 off-center from the LA of endoprobe 100, attached to inner tube 130. In some configurations, distal element 970, attached to outer tube 140, is placed off-center from the LA of endoprobe 100. Some embodiments may place proximal and distal elements 960 and 970 concentric about the LA of endoprobe 100, but with either one of the proximal or distal elements having an optical axis off-center from the LA of endoprobe 100. For example, a rod style GRIN lens may be built with the optical axis offset from the LA of endoprobe 100 and used as a proximal or a distal element in an eccentric configuration. In some embodiments a conventional or aspheric lens may be used as one of elements 960 or 970, and an eccentricity may be provided by grinding the lens so that the optical axis is off-center relative to the LA of endoprobe 100. Further embodiments may include a conventional, aspheric, or a GRIN lens cut in a cylindrical shape having a geometrical center offset from the center of the cylinder.

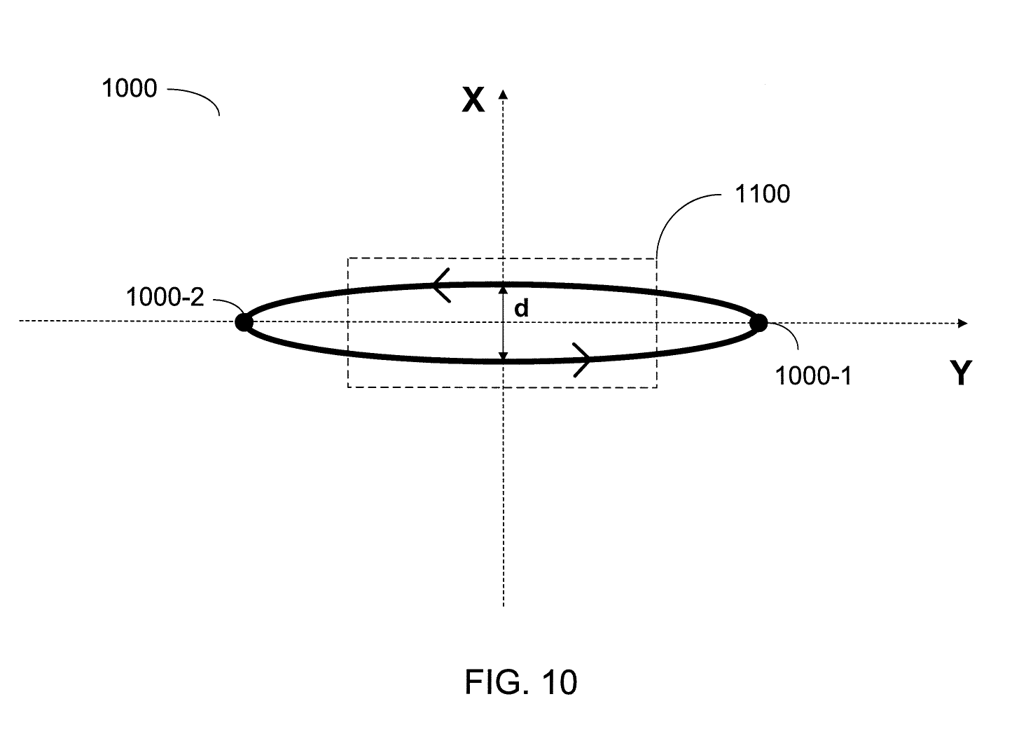

FIG. 10 illustrates a scanning trajectory 1000 of an optical beam, according to some embodiments. Forward and backward scans are offset by a distance ‘d’ along the X-axis, providing two B-scans per 180 degree rotation. Thus doubling the speed for collecting a C-scan image. End points 1000-1 and 1000-2 are coincident (for registration purposes). Portion 1100 of scanning trajectory 1000 may be the imaging portion of the trajectory, according to some embodiments. That is, in such embodiments, portion 1100 may be the portion of trajectory 1000 that collects imaging data in an endoprobe consistent with the present disclosure. Use of an eccentric rotational mechanism as disclosed herein may result in a decreased scan range in scanning trajectory 1000.

Embodiments of the invention described above are exemplary only. One skilled in the art may recognize various alternative embodiments from those specifically disclosed. Those alternative embodiments are also intended to be within the scope of this disclosure.

What is claimed is:

1. A microsurgical endoprobe comprising
a hand-piece comprising a motor;
a cannula assembly coupled to the hand-piece;
the cannula assembly comprising:
a first tube able to rotate about a longitudinal axis;
a second tube within the first tube, able to rotate within the first tube;
a distal optical element attached to the first tube; and
a proximal optical element attached to the second tube; wherein
the motor is coupled to provide a reciprocating rotational motion to at least one of the second tube and the first tube for an arc period.

2. The endoprobe of claim 1 wherein the distal optical element and the proximal optical element comprise a GRIN (gradient index) lens.

3. The endoprobe of claim 2 wherein the GRIN lenses have a face cut at a non-orthogonal angle relative to a longitudinal axis.

4. The endoprobe of claim 1 wherein at least one of the distal optical element and the proximal optical element comprise a prism.

5. The endoprobe of claim 1 wherein the reciprocating motion includes
a first cycle rotating the first tube in a first direction and counter-rotating the second tube; and
a second cycle rotating the second tube in the first direction and counter-rotating the first tube.

6. The endoprobe of claim 1 wherein the arc period is less than or equal to 180 degrees.

7. The endoprobe of claim 1 wherein at least one of the distal optical element and the proximal optical element has an optical axis concentric with the longitudinal axis.

8. The endoprobe of claim 1 wherein at least one of the distal optical element and the proximal optical element has an optical axis eccentric to the longitudinal axis.

9. An optical cannula assembly comprising
an outer tube able to rotate about a longitudinal axis;
an inner tube within the outer tube, able to rotate within the outer tube;
a distal optical element attached to the outer tube; and
a proximal optical element attached to the inner tube; wherein
a proximal end of the cannula assembly is configured to engage a mechanical actuator to provide a reciprocating rotational motion to at least one of the outer tube and the inner tube for an arc period.

10. The cannula assembly of claim 9 wherein the outer tube has a first cross-sectional area and the distal optical element has a second cross-sectional area, the second cross-sectional area being greater than 50% of the first cross-sectional area.

11. The cannula assembly of claim 9 wherein the outer tube has a tissue engaging surface.

12. A method for using a microsurgical endoprobe having a cannula assembly, comprising:
inserting the microsurgical endoprobe into a tissue, the microsurgical endoprobe having a cannula assembly with a proximal element and a distal element coupled to a mechanical actuator;
rotating at least one of the proximal element and the distal element in a first direction for an arc period;
performing a microsurgical procedure on the tissue; and
rotating the at least one of the proximal element and the distal element in a second direction opposite to the first direction for the arc period.

13. The method of claim 12 wherein the arc period is less than or equal to 180 degrees.

14. The method of claim 12 wherein the rotating at least one of the proximal element and the distal element comprises rotating the proximal element for the arc period and counter-rotating the distal element for the arc period.

15. The method of claim 12 wherein the inserting the microsurgical endoprobe into the tissue comprises engaging at least one of the first tube and the second tube with the tissue.

16. The method of claim 12 wherein the arc period is less than or equal to 180 degrees.

17. The method of claim 12 wherein the arc period is less than or equal to 90 degrees.

18. The method of claim 12 wherein the microsurgical procedure is an Optical Coherence Tomography (OCT) scan.

19. The method of claim 12 wherein the microsurgical procedure includes distributing therapeutic laser pulses across the retina.

20. The method of claim 12 wherein the microsurgical procedure is pan retinal photocoagulation.
